(19) United States
(12) Patent Application Publication
(10) Pub. No.: US 2008/0255132 A1
(43) Pub. Date: Oct. 16, 2008

(54) Combination Therapy Comprising the Use of Et-743 and Paclitaxel for Treating Cancer

(76) Inventors: Eric Rowinsky, San Antonio, TX (US); Quincy Siu-Ching Chu, San Antonio, TX (US); Jose Maria Jimeno Donaque, Madrid (ES); Luis Lopez Lazaro, Madrid (ES)

Correspondence Address: KING & SPALDING, 1185 AVENUE OF THE AMERICAS, NEW YORK, NY, 10036-4003, US

(21) Appl. No.: 10/579,130

(22) PCT Filed: Oct. 28, 2004
(86) PCT No.: PCT/US04/35779

§ 371 Date: Aug. 13, 2007

Related U.S. Application Data
(60) Provisional application No. 60/520,330, filed on Nov. 14, 2003.

(51) Int. Cl. A61K 31/4985; A61P 35/00

(52) U.S. Cl. 514/250

(57) ABSTRACT

Methods of treating a human body for cancer are provided. In one aspect, an effective therapeutic amount of paclitaxel is administered in combination with ET-743 in a dose range between 0.5 and 1 mg/m². In a related aspect, an effective therapeutic amount of ET-743 is administered in combination with paclitaxel in a dose range between 80 and 140 mg/m².

The invention relates to a combination therapy, more particularly a combination therapy for cancer.

### FIELD OF THE INVENTION

The present invention is directed to the use of ecteinascidin 743 and products containing this compound for cancer therapy. In particular the present invention is directed to the use of ecteinascidin 743 in combination with paclitaxel for the treatment of cancer.

### BACKGROUND OF THE INVENTION

Cancer comprises a group of malignant neoplasms that can be divided into two categories, carcinoma, comprising a majority of the cases observed in the clinics, and other less frequent cancers, which include leukemia, lymphoma, central nervous system tumors and sarcoma. Carcinomas have their origin in epithelial tissues while sarcomas develop from connective tissues and those structures that had their origin in mesoderm tissues. Sarcomas can affect, for instance, muscle or bone and occur in the bones, bladder, kidneys, liver, lung, parotid, spleen, etc.

Cancer is invasive and tends to metastasise to new sites. It spreads directly into surrounding tissues and also may be disseminated through the lymphatic and circulatory systems.

Many treatments are available for cancer, including surgery and radiation for localised disease, and drugs. However, the efficacy of available treatments on many cancer types is limited, and new, improved forms of treatment showing clinical benefit are needed.

This is especially true for those patients presenting with advanced and/or metastatic disease. It is also true for patients relapsing with progressive disease after having been previously treated with established therapies for which further treatment with the same therapy is mostly ineffective due to acquisition of resistance or to limitations in administration of the therapies due to associated toxicities.

Chemotherapy plays a significant part in cancer treatment, as it is required for treatment of advanced cancers with distant metastasis and often helpful for tumor reduction before surgery, and many anti-cancer drugs have been developed based on various modes of action.

The most commonly used types of anticancer agents include: DNA-alkylating agents (for example, cyclophosphamide, ifosfamide), antimetabolites (for example, methotrexate, a folate antagonist, and 5-fluorouracil, a pyrimidine antagonist), microtubule disrupters (for example, vincristine, vinblastine, paclitaxel), DNA intercalators (for example, paclitaxel, daunomycin, cisplatin), and hormone therapy (for example, tamoxifen, flutamide). The ideal antineoplastic drug would kill cancer cells selectively, with a wide therapeutic index relative to its toxicity towards non-malignant cells. It would also retain its efficacy against malignant cells, even after prolonged exposure to the drug. Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents.

The ecteinascidins (herein abbreviated ETs) are exceedingly potent antitumor agents isolated from the marine tunicate Ecteinascidia turbinata. Several ecteinascidins have been reported previously in the patent and scientific literature. See, for example U.S. Pat. No. 5,089,273, which describes compounds extracted from the tropical marine invertebrate Ecteinascidia turbinata, and designated therein as ecteinascidins 729, 743, 745, 759A, 759B and 770. These compounds are useful as antibacterial and/or antitumor agents in mammals. U.S. Pat. No. 5,478,932 describes ecteinascidins isolated from the Caribbean tunicate Ecteinascidia turbinata, which provide in vivo protection against P388 lymphoma, B16 melanoma, M5076 ovarian sarcoma, Lewis lung carcinoma, and the LX-1 human lung and MX-1 human mammary carcinoma xenografts.

One of the ETs, ecteinascidin-743 (ET-743), is a novel tetrahydroisoquinoline alkaloid with considerable antitumor activity in murine and human tumors in vitro and in vivo, and is presently in clinical trials. ET-743 possesses potent antineoplastic activity against a variety of human tumor xenografts grown in athymic mice, including melanoma and ovarian and breast carcinoma.

A clinical development program of ET-743 in cancer patients was started with phase I studies investigating 1-hour, 3-hour, 24-hour and 72-hour intravenous infusion schedules and a 1 hour daily×5 (d×5) schedule. Promising responses were observed in patients with sarcoma and breast and ovarian carcinoma. Therefore this new drug is currently under intense investigation in several phase II clinical trials in cancer patients with a variety of neoplastic diseases.

Further detail on the use of ET-743 for the treatment of the human body for cancer is given in WO 0069441, incorporated herein by reference in its entirety. At pages 8 and 9, this patent specification indicates that ET-743 may be employed in a combination therapy with another drug. A list of candidates for the other drug is given, and mentions paclitaxel.

A recent review of ET-743, its chemistry, mechanism of action and preclinical and clinical development can be found in Kesteren, Ch. Van et al., 2003, Anti-Cancer Drugs, 14 (7), pages 487-502: “Yondelis (trabectedin, ET-743): the development of an anticancer agent of marine origin”, and references therein.

Combination therapy using drugs with different mechanisms of action is an accepted method of treatment which helps prevent development of resistance by the treated tumor. In vitro activity of ET-743 in combination with other anticancer agents has been studied, see for example WO 02 36135, incorporated herein by reference in its entirety.

In particular, WO 0236135 mentions the combination of ET-743 with paclitaxel. An effect is noted in tests on animal models.

Takahashi et al. in Clinical Cancer Research, 7: 3251-3257, 2001, report on Sequence-dependent Enhancement of Cytotoxicity Produced by Ecteinascidin 743 (ET-743) with Doxorubicin or Paclitaxel in Soft Tissue Sarcoma Cells. They used two soft tissue sarcoma cell lines, and report that ET-743 and paclitaxel result in strong cytotoxic synergism when paclitaxel is administered before ET-743, but less than additive toxicity when ET-743 is added concomitantly or before paclitaxel.

In Cancer Research 62: 6909-6915, 2002, Takahashi et al. describe work on Sequence-dependent Synergistic Cytotoxicity of Ecteinascidin 743 and Paclitaxel in Human Breast Cancer Cell Lines in vitro and in vivo. They found that pretreatment with paclitaxel before ET-743 was the most effective combination in three breast cancer cell lines, and that sequential treatment with paclitaxel followed by ET-743 increases the antitumor effects in nude mice bearing carcinoma xenografts, without increasing toxicity.

It is an object of the invention to provide an efficacious combination treatment of cancer based on ET-743 with paclitaxel.

### SUMMARY OF THE INVENTION

According to the present invention, we provide a combination therapy for the treatment of cancer in humans which employs ecteinascidin 743 and paclitaxel, using a cyclical dosing protocol. Typical dosing protocols for the combination therapy are provided. From phase I clinical trials, we have determined that a combination of ET-743 and paclitaxel in humans is tolerable and feasible, and that at the dosage and regimens given there is evidence of antitumor activity.

We also provide a method of treating a cancer patient, which comprises administering ET-743 and paclitaxel in a specified sequence. The ET-743 and paclitaxel are suitably administered on the basis of a predetermined cycle.

We further provide the use of ET-743 in the preparation of a medicament for carrying out the method of treatment. We also provide the use of paclitaxel, in the preparation of a medicament for carrying out the method of treatment. We provide the use of ET-743 and paclitaxel, in the preparation of a medicament for carrying out the method of treatment.

### DETAILED DESCRIPTION

ET-743 is a natural compound represented by the following formula:

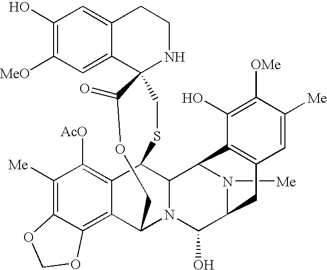

As used herein, the term “ET-743” also covers any pharmaceutically acceptable salt, ester, solvate, hydrate or a prodrug compound which, upon administration to the recipient is capable of providing (directly or indirectly) the compound ET-743. The preparation of salts and other derivatives, and prodrugs, can be carried out by methods known in the art.

ET-743 is typically supplied and stored as a sterile lyophilized product, with ET-743 and excipient in a formulation adequate for therapeutic use, in particular a formulation containing mannitol and a phosphate salt buffered to an adequate pH.

The dose of ET-743 will be selected according to the dosing schedule, having regard to the existing data on Dose Limiting Toxicity, on which see for example the incorporated WO patent specifications, and also see Kesteren, Ch. Van et al., 2003, Anti-Cancer Drugs, 14 (7), pages 487-502: “Yondelis (trabectedin, ET-743): The development of an anticancer agent of marine origin”. This article is incorporated herein in full by specific reference.

For a single administration of ET-743 at around the start of each cycle, we prefer a dose in the range 0.2 to 2 mg/m², more preferably 0.4 to 1.4 mg/m², most preferably 0.5 to 1 mg/m². In one embodiment the dose of ET-743 is about 0.58-0.9 mg/m². At this stage, we currently prefer a dose of about 0.65 mg/m², about 0.775 mg/m² or about 0.9 mg/m². Lower amounts are suitable where there is repeat dosing on a weekly or daily schedule.

As noted in the incorporated article by Kesteren, the combination of ET-743 with dexamethasone gives unexpected advantages. It has a role in hepatic prophylaxis. We therefore prefer to administer dexamethasone to the patient, typically at around the time of infusing the ET-743. For example, we prefer to give dexamethasone before ET-743 on the same day. The administration of dexamethasone can be extended, for example to one or more days preceding or following ET-743.

Paclitaxel is used for the treatment of many cancers, including for example, metastatic breast cancer, metastatic ovarian cancer, Kaposi's sarcoma, head and neck cancer, non-small cell lung cancer, small cell lung cancer, and bladder cancer.

The dosage amount of paclitaxel is preferably in the range from 50 to 200 mg/m², more preferably 60 to 150 mg/m². At this stage, we currently prefer a dose of about 80 mg/m², about 120 mg/m² or about 140 mg/m².

In the present invention, ET-743 and paclitaxel are administered in combination as part of an antitumor therapy. It is preferred to administer the combination by infusion. ET-743 and paclitaxel may be provided as separate medicaments for administration at the same time or at different times. Preferably, ET-743 and paclitaxel are provided as separate medicaments for administration at different times. When administered separately and at different times, it is preferable to administer paclitaxel followed by ET-743.

The infusing step is typically repeated on a cyclic basis, which may be repeated as appropriate over for instance 1 to 35 cycles. The cycle includes a phase of infusing the combination, and usually also a phase of not infusing the combination. Typically the cycle is worked out in weeks, and thus the cycle normally comprises one or more weeks of drugs infusion phase, and one or more weeks to complete the cycle. Usually a cycle can be from 1 to 6 weeks. In one embodiment a cycle of 2 weeks is preferred. The infusion phase can itself be a single administration in each cycle of say 1 to 72 hours, more usually about 1, 3 or 24 hours. When paclitaxel and ET-743 are provided as separate medicaments for administration at different times, the infusion times for each may differ. Infusion times for paclitaxel are generally up to 6 hours, more preferably 1-3 hours, with 1 hour most preferred. Infusion times for ET-743 are generally up to 24 hours, more preferably about 1, about 3 or about 24 hours. Short infusion times which allow treatment to be carried out without an overnight stay in hospital are especially desirable. Thus, for example, a single administration of paclitaxel on day 1 followed by a single administration of ET-743 on day 2 of a 2 week cycle is preferred. Other administration protocols can be designed having regard to this embodiment.

Premedication and supportive medication can be given. Mention has already been made of dexamethasone with the ET-743, but further options include dexamethasone premed for paclitaxel, diphehydramine premed for paclitaxel, ranitidine premed for paclitaxel, 5-HT₃ antagonist premed or supportive medication for ET-743.

Depending on the type of tumor and the developmental stage of the disease, the treatments of the invention are useful in preventing the risk of developing tumors, in promoting tumor regression, in stopping tumor growth and/or in preventing metastasis. In particular, the method of the invention is suited for human patients, especially those who are relapsing or refractory to previous chemotherapy. First line therapy is also envisaged.

Preferably, the combination therapy is used according to the above schedules and dosages for the treatment of sarcoma, osteosarcoma, ovarian cancer, breast cancer, melanoma, colorectal cancer, mesothelioma, renal cancer, endometrial cancer and lung cancer. Most preferably the patients are sarcoma patients, especially those with a soft tissue sarcoma. Ovarian cancer and breast cancer are also preferably suited for the combination therapy.

In a further aspect of the present invention, a medical kit for administering ET-743 in combination with paclitaxel is provided, comprising printed instructions for administering ET-743 according to the dosing schedules set forth above, and a supply of ET-743 in dosage units for at least one cycle, wherein each dosage unit contains the appropriate amount of ET-743 for the treatments as defined above and a pharmaceutically acceptable carrier.

Although guidance for the dosage is given above, the correct dosage of the compound will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

### EXAMPLE

### Phase I Clinical Trial

A phase I trial combining paclitaxel and trabectedin was performed. The objective of this study was to determine the maximum tolerated dose, the safety profile and the tolerability of the sequential administration of paclitaxel as a 1-hour infusion followed by ET-743 as a 3-hour infusion, 24 hours later, every 2 weeks in patients with advanced solid tumors.

All patients enrolled in the study fulfilled the following criteria:
Histological diagnosis of advanced solid tumor,
Had at least 4 weeks since chemotherapy (6 weeks since nitrosureas and mitomycin C), immunotherapy, hormonal therapy or any anti-tumoral therapy or investigational agents and wide-field radiation involving >25% of bone marrow reserve,
Age of at least 18,
ANC≧1500/mm³, PLT≧100,000/mm³ and Hgb≧8.5 g/dL,
Adequate renal function: calculated creatinine clearance ≧50 ml/min,
Adequate hepatic function: albumin≧2.5 g/dL, total bilirubin≦10.0×ULN, AST & ALT≦3×ULN, total ALKP≦1.5×ULN,
Central venous assess or willing to undergo placement of a central venous assess,
Eastern Cooperative Oncology Group (ECOG) Performance Status (PS) score of 0-2,
Life expectancy >3 months,
Treated or asymptomatic CNS metastases,
Absence of any concurrent serious medical illnesses, which may increase patient's risk during therapy,
Absence of prior ET-743 exposure or documented allergy to ET-743,
Had ≦1 peripheral neuropathy.

In this study, each cohort of at least 3 patients was treated with escalating doses of ET-743 and paclitaxel. The treatment plan was the following:

### 1. Paclitaxel Administration:

Premedication: Dexamethasone 20 mg i.v., diphenhydramine 50 mg i.v. and ranitidine 50 mg i.v. 30-60 minutes prior to the administration of paclitaxel,
Paclitaxel was administered as a 1-hour infusion on day 1 of each cycle, except in cycle 1 in which it was administered on day −7 (7 days before cycle 1 day 1).

### 2. ET-743 Administration:

Premedication: Dexamethasone 10 mg i.v. and 5-HT₃ antagonist i.v. 30-60 minutes prior to the administration of ET-743,
ET-743 is administered as a 3-hour infusion on day 2 of each cycle through a central venous catheter,
Supportive medication: 5-HT₃ antagonist was given starting 24 hours after ET-743 for 3 days.

Dose-limiting toxicity (DLT) was defined during the first 2 cycles of treatment as:
ANC<500 μL for more than 5 days,
ANC<1000 μL with fever (≧38.5° C.)
PLT<25,000/μL,
Grade 3 for >7 days or grade 4 transaminitis that leads to delay of a cycle,
Any grade 3 or 4 non-hematological toxicity except inadequately treated nausea or vomiting,
Delay in the initiation of subsequent cycle for >1 week due to toxicity,
Any grade 4 transaminitis.

A total of 18 patients were enrolled to the first 5 dose levels. The patient characteristics are tabulated as below:

| | |
|---|---|
| Median Age (Range) | 39 (18-67) |
| Male:Female | 11:7 |
| Heavily:Lightly Pre-treated | 13:5 |
| Median Number of Prior Chemotherapy, | 3 (1-5) |
| Regimens (Range) | |
| No. of Patients with Adjuvant Chemotherapy | 8 |
| No. of Patients with Neoadjuvant Chemotherapy | 4 |
| Prior Ifosphamide/Doxorubicin | 13 |
| No. of Patients with Prior Autologous BMT | 2 |
| No. of Patients with Prior Radiation | 6 |
| Type of Tumor | |
| Soft Tissue Sarcoma | 17 |
| Melanoma | 1 |
| Median No. of Cycles of ET-743/paclitaxel (Range) | 4 (1-28) |

Table 1 show the number of patients exposed in each doses of Paclitaxel/ET-743 and the dose limiting toxicities observed.

TABLE1

| Doses of Paclitaxel | Number of Patient | |
|---|---|---|
| (mg/m²)/ET-743 (mg/m²) | in the Cohort | Dose-limiting Toxicity |
| Cohort 1: 80/0.525 | 3 | Nil |
| Cohort 2: 80/0.58 | 3 | Nil |
| Cohort 3: 120/0.58 | 6 | Delay of Cycle 2 for >1 |
| | | week due to ANC <1.5 |
| Cohort 4: 120/0.65 | 3 | Nil |
| Cohort 5: 120/0.775 | 3 | Delay of Cycle 3 for >1 |
| | | week due to ANC <1.5 |

Table 2 shows the frequently reported drug-related toxicities. In order to define the toxicity grade, NCI common criteria is used.

TABLE 2

| Toxicity | Cohort 1 | Cohort 2 | Cohort 3 |
|---|---|---|---|
| Leukopenia | Gr 1 (1) Gr 2 (1) | Gr 1 (2) Gr 2 (1) | Gr 1 (1) Gr 4 (1) |
| Neutropenia | Gr 1 (1) Gr 2 (1) | Nil | Gr 2 (1) Gr 4 (1) |
| Anemia | Gr 1 (2) Gr 2 (1) | Gr 2 (1) | Gr 1 (3) Gr 2 (1) |
| AST/ALT | Gr 1 (1) G2 (1) | Gr 1 (1) G2 (1) | Gr 1 (1) G2 (1) |
| elevation | | | |
| Peripheral | Gr 1 (1) | Gr 1 (2) | Nil |
| Neuropathy | | | |
| Fatigue | Gr 1 (1) | Nil | Gr 1 (5) |
| Nausea/ | Nil | Gr 1 (2) | Gr 1 (3) |
| vomiting | | | |
| Myalgia | Nil | Nil | Gr 1 (2) |
| Alopecia | Gr 4 (1) | Nil | Gr 4 (1) |
| Elevation of | Nil | Gr 1 (1) | Gr 1 (2) |
| Creatinine | | | |
| Elevation of | Gr 2 (1) | Nil | Nil |
| CK | | | |

| Toxicity | Cohort 4 | Cohort 5 |
|---|---|---|
| Leukopenia | Gr 1 (1) Gr 2 (1) | Gr 2 (1) Gr 3 (1) |
| Neutropenia | Gr 2 (1) Gr 4 (1) | Gr 2 (1) Gr 4 (1) |
| Anemia | Gr 1 (1) Gr 2 (1) | Nil |
| AST/ALT | Gr 1 (1) G2 (1) | Gr 1 (1) |
| elevation | | |
| Peripheral | Gr 1 (1) | Gr 1 (1) |
| Neuropathy | | |
| Fatigue | Gr 1 (1) | Gr 1 (2) |
| Nausea/vomiting | Nil | Gr 2 (2) |
| Myalgia | Nil | Gr 2 (1) |
| Alopecia | Nil | Nil |
| Elevation of | Nil | Nil |
| Creatinine | | |
| Elevation of CK | Gr 2 (1) | Nil |

Regarding the antitumoral activity of the combination, Positive Responses (PR) were observed in one doxorubicin/ifosphamide-resistant liposarcoma patient after Cycle 8 until Cycle 16 and in one primitive neuroectodermal tumor (pNET) patient after Cycle 4. In addition, Prolonged Stabilisation Disease (SD>24 weeks) were observed in one primitive neuroectodermal tumor (pNET) patient (28 cycles), in one melanoma patient (26 cycles) and in one liposarcoma patient (20 cycles)

In conclusion, except one heavily pre-treated leiomyosarcoma in cohort 5 experienced DLT with delay of Cycle 3 due to ANC<1.5 for more than 1 week, this sequential combination of paclitaxel and ET-743 every 2 week is very well tolerated.

Some anti-tumor activity is observed in one doxorubicin/ifosphamide-resistant liposarcoma and in one pNET and prolonged stable disease is also observed in one pNET, in one melanoma and in one liposarcoma.

1. A method of treating a human body for cancer comprising administering an effective therapeutic amount of paclitaxel, in combination with ET-743 in a dose range between 0.5 and 1 mg/m² for ET-743.

2. A method of treating a human body for cancer comprising administering an effective therapeutic amount of ET-743, in combination with paclitaxel in a dose range between 80 and 140 mg/m² for paclitaxel.

3. The method according to claim 1 or 2, wherein paclitaxel and ET-743 are provided as separate medicaments for administration at different times.

4. The method according to claim 3, wherein paclitaxel is administered prior to the administration of ET-743.

5. The method according to claim 4, wherein paclitaxel and ET-743 are administered by intravenous injection.

6. The method according to claim 5, wherein the infusion time for intravenous injection is up to 3 hours for paclitaxel and up to 24 hours for ET-743.

7. The method according to claim 6, wherein the infusion time for intravenous injection is about 1 hour for paclitaxel and about 3 hours for ET-743.

8. The method according to claim 7, where the infusions are carried out at an interval of 1 to 6 weeks.

9. The method according to claim 8, wherein the infusions are carried out at an interval of 2 weeks.

10. The method according to claim 9, wherein paclitaxel is administered in a dosage of up to 120 mg/m², followed by ET-743 which is administered in a dosage of up to 0.775 mg/m².

11. The method according to claim 10, wherein paclitaxel is administered in a dosage about 120 mg/m², followed by ET-743 which is administered in a dosage about 0.650 mg/m².

12. A method according to any preceding claim, in which the patient has a cancer selected from sarcoma, osteosarcoma, ovarian cancer, breast cancer, melanoma, colorectal cancer, mesothelioma, renal cancer, endometrial cancer and lung cancer.

13. A method according to claim 12, in which the patient has a cancer selected from sarcoma, ovarian cancer and breast cancer.

14. The use of paclitaxel in the preparation of a medicament for a method according to any of claims 1 to 13.

15. The use of ET-743 in the preparation of a medicament for a method according to any of claims 1 to 13.

16. A medical kit for administering ET-743 in combination with paclitaxel, comprising a supply of ET-743 in dosage units for at least one cycle, wherein each dosage unit contains the appropriate amount of ET-743 for the treatments and a pharmaceutically acceptable carrier, and printed instructions for administering ET-743 according to a dosing schedule.
